(19) United States
(12) Patent Application Publication
(10) Pub. No.: US 2018/0256601 A1
(43) Pub. Date: Sep. 13, 2018

(54) URSODEOXYCHOLIC ACID AND BRAIN DISORDERS

(71) Applicants: Giampiero Bartolucci, Hamilton (CA); Michael Zablocki, Hamilton (CA)

(72) Inventors: Giampiero Bartolucci, Hamilton (CA); Michael Zablocki, Hamilton (CA)

(21) Appl. No.: 15/760,080

(22) PCT Filed: Sep. 16, 2016
(86) PCT No.: PCT/US16/52071

§ 371 Date: Mar. 14, 2018

Related U.S. Application Data
(60) Provisional application No. 62/219,186, filed on Sep. 16, 2015.

(51) Int. Cl. A61K 31/575; A61K 9/00; A61K 47/34; A61K 9/06; A61K 47/46; A61K 47/10; A61K 47/32; A61K 47/18; A61K 9/107; A61K 47/40; A61P 25/00

(52) U.S. Cl. CPC A61K 31/575; A61K 9/0014; A61K 47/34; A61K 9/06; A61K 47/46; A61P 25/00; A61K 47/32; A61K 47/18; A61K 9/0019; A61K 9/107; A61K 47/40; A61K 47/10

(57) ABSTRACT

The present disclosure provides, among other things, uses for ursodeoxycholic acid or analogs or conjugates or derivatives thereof, for example for ameliorating the effects of brain disorders, neurodevelopmental disorders, and neuropsychiatric disorders, and compositions relating thereto.

### BACKGROUND

There is long felt and significant need for therapies to treat brain disorders, neurodevelopmental disorders, and neuropsychiatric disorders.

### SUMMARY

The present invention is in the field of medicine and relates to methods, compositions, and the use of those methods and compositions for treating brain disorders, neurodevelopmental disorders, and/or neuropsychiatric disorders, for example Autism Spectrum Disorder.

### Definitions

Administration: As used herein, the term “administration” typically refers to the administration of a composition to a subject or system. Those of ordinary skill in the art will be aware of a variety of routes that may, in appropriate circumstances, be utilized for administration to a subject, for example a human. For example, in some embodiments, administration may be ocular, oral, parenteral, topical, etc. . . . . In some particular embodiments, administration may be bronchial (e.g., by bronchial instillation), buccal, dermal (which may be or comprise, for example, one or more of topical to the dermis, intradermal, interdermal, transdermal, etc), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Adult: As used herein, the term “adult” refers to a human eighteen years of age or older. In some embodiments, a human adult has a weight within the range of about 90 pounds to about 300 pounds.

Agent: In general, the term “agent”, as used herein, may be used to refer to a compound or entity of any chemical class including, for example, a polypeptide, nucleic acid, saccharide, lipid, small molecule, metal, or combination or complex thereof. In appropriate circumstances, as will be clear from context to those skilled in the art, the term may be utilized to refer to an entity that is or comprises a cell or organism, or a fraction, extract, or component thereof. Alternatively or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some cases, the term “agent” may refer to a compound or entity that is or comprises a polymer; in some cases, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety.

Amelioration: as used herein, refers to the prevention, reduction or palliation of a state, or improvement of the state of a subject. Amelioration includes, but does not require complete recovery or complete prevention of a disease, disorder or condition.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: Two events or entities are “associated” with one another, as that term is used herein, if the presence, level and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Baby: As used herein, the term “baby” refers to a human under two years of age. Typical body weights for a baby ranges from 3 pounds up to 20 pounds.

Brain Disorder: As used herein, the term “brain disorder” refers to a condition in which brain activity produces behavior which causes a person to have a significantly reduced ability to function well in his or her life and to adapt to change in environment, social environment, new tasks or poses a threat to his/her well-being and/or the well-being of others. In some embodiments, a brain disorder derives primarily from a functional disorder of the brain which may cause cognitive and behavioral patterns and habits that, over time, cause a secondary effect on brain activity. In some embodiments, a brain disorder is or comprises a lesion, such as a thrombosis or hemorrhage; a tumor; an infectious or inflammatory brain illness; and/or a degenerative brain condition, such as Alzheimer's or Parkinson's disease.

Child: As used herein, the term “child” refers to a human between two and 18 years of age. Body weight can vary widely across ages and specific children, with a typical range being 30 pounds to 150 pounds. In some embodiments, a child is a pediatric patient.

Combination therapy: As used herein, the term “combination therapy” refers to those situations in which a subject is simultaneously exposed to two or more therapeutic regimens (e.g., two or more therapeutic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agents or modalities to a subject receiving the other agents or modalities in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

Composition: Those skilled in the art will appreciate that the term “composition”, as used herein, may be used to refer to a discrete physical entity that comprises one or more specified components. In general, unless otherwise specified, a composition may be of any form—e.g., gas, gel, liquid, solid, etc.

Comprising: A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Dosage form or unit dosage form: Those skilled in the art will appreciate that the term “dosage form” may be used to refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

Dosing regimen: Those skilled in the art will appreciate that the term “dosing regimen” may be used to refer or a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount In some embodiments, a dosing regimen is correlated with a desired or beneficial outcome when administered across a relevant population (i.e., is a therapeutic dosing regimen).

Human: In some embodiments, a human is an embryo, a fetus, an infant, a child, a teenager, an adult, or a senior citizen.

Improve,” “increase” or “reduce: As used herein or grammatical equivalents thereof, indicate values that are relative to a baseline measurement, such as a measurement in the same individual prior to initiation of a treatment described herein, or a measurement in a control individual (or multiple control individuals) in the absence of the treatment described herein. In some embodiments, a “control individual” is an individual afflicted with the same form of disease or injury as an individual being treated.

Neurodevelopmental disorders: As used herein, the term “neurodevelopmental disorders” refers to a group of conditions with onset in the developmental period from conception to adolescence. Neurodevelopmental disorders typically manifest early in development and are characterized by developmental deficits that produce impairments of personal, social, academic, or occupational functioning. The range of developmental deficits associated with any particular neurodevelopmental disorder often can vary from very specific limitations of learning or control of executive functions to global impairments of social skills or intelligence. In some embodiments, neurodevelopmental disorders may co-occur; for example, individuals with autism spectrum disorder may have intellectual disability (intellectual developmental disorder); individuals with attention-deficit/hyperactivity disorder (ADHD) may also have a specific learning disorder, etc. In some embodiments, the clinical presentation of a neurodevelopmental disorder may include one or more symptoms of deviant, unusual behaviors, as well as one or more deficits and/or delays in achieving expected milestones. For example, autism spectrum disorders are typically diagnosed only when the characteristic deficits of social communication are accompanied by excessively repetitive behaviors, restricted interests, and insistence on sameness. In some embodiments, a neurodevelopmental disorder may be a traumatic developmental disorder. Typically, a traumatic developmental disorder involves exposure after birth and during early development to one or more interpersonally, socially, and physiologically traumatic events leading to developmental delay and/or other abnormality that may not be not obviously attributable to another factor (e.g., to a physical defect or damage). Those skilled in the art will be familiar with so-called “Adverse Childhood Experiences” (ACE) research and its approach to documenting severity and/or consequences of such events. Without wishing to be bound by any particular theory, it is noted that such research proposes that ACEs disrupt neurodevelopment, resulting in created social, emotional, and/or cognitive impairments that may lead to disease, disability, social problems, and/or death (See, for example, Centers for Disease Control and Prevention (CDC) “ACE Pyramid”; see also Felitti et al Am J Prev Med 14:245, May 1998). Examples of ACEs include but are not limited to multiple types of abuse; neglect; violence between parents or caregivers; other kinds of serious household dysfunction such as alcohol and substance abuse; and peer, community and collective violence. Considerable and prolonged exposure to ACEs may disrupt early brain development and/or compromise nervous and/or immune system functioning (See, for example, World Health Organization (WHO) “ACE-IQ Rationale”). ACEs can be assessed and evaluated using a variety of questionnaires, including those provided by, for example, the CDC and/or the WHO; exemplary such questionnaires include the Adverse Childhood Experiences International Questionnaire (ACE-IQ), Family Health History Questionnaire, Health Appraisal Questionnaire, and the BRFSS Adverse Childhood Experiences Module. In some embodiments, a subject showing a developmental disorder and/or delay associated with significant ACE score may also show symptoms of a diagnostic category. Those skilled in the art will appreciate that typical symptoms of ACE include early child abuse, either interpersonal or sexual, but that these symptoms are not the only ACE events that may lead a traumatic developmental disorder. In some embodiments, a traumatic developmental disorder is differentiated from perinatal illnesses or medical complications associated with negative maternal factors (such as maternal rheumatoid arthritis in ASD and fetal alcohol syndrome).

Neuropsychiatric disorders: As used herein, the term “neuropsychiatric disorders” refers to brain dysfunctions which cause behavioral or cognitive disorders, for example involving misinterpretation of communication and/or of social interactions; disorders of communication in social contexts; and/or problems in adaptation to change, for example in physical and/or social environment and/or in activities of daily living or social routine. In some embodiments, a neuropsychiatric disorder may be characterized by one or more maladaptations that may appear in late childhood or later in life; in some embodiments, onset may occur in late adulthood or early old age (e.g., at an age within the range of early fifties to early seventies. In some embodiments, a neuropsychiatric disorder may be characterized by one or more social disturbances of communication and/or interpretation by the person affected by the behavior of others, as in paranoid states, and/or may be accompanied by auditory hallucinations or, in some (less common) embodiments, by hallucinations of one or more other sensory systems, as in the “psychosis” called schizophrenias. In some embodiments, a neuropsychiatric disorder may present with a depressive disorder such as major depression; in some such embodiments, the depressive disorder may alternate with periods of elation, grandiosity and/or loss of judgment, such as may be understood to characterize manic or hypomanic episodes as in bipolar disorders or as they are today identified in non-categorical identification as manic-depressive psychoses. In some embodiments, a neuropsychiatric disorder may be identified by the DSM-5 as Psychotic Processes occurring in two major broad categories of psychoses, currently limited to Schizophrenic and Manic-Depressive psychoses. Those skilled in the art will appreciate that DSM-5 currently relies on psychotic processes as described to identify the patients as suffering from a particular disorder or condition, and furthermore will appreciate that such identification relies upon identification and description of features considered relevant in a clinical examination according to judgment of the Senior Inventor (GB). Over time, an algorithm may be developed, for example to express numerically the clinical judgment used and/or describe baseline and change in treatment protocols. Thus, description of these characteristics in subjects can lead to categorical, diagnostic classifications according to the DSM-IV or -5; in some embodiments, however, these diagnostic categories may be noted without being relied upon for identification of the subjects nor reported as criteria for stratification of the results. In some embodiments, a neuropsychiatric disorder may be identified in accordance with the present invention by categorical diagnostic identification as Post Traumatic Stress Disorder in adults or generally fully developed individuals exposed to violent forces as part of accident (e.g., motor vehicle accidents) or war related events, social and physical abuse with consequences described in syndromes of Post Traumatic Stress Disorder, at times, but not always associated with evidence of traumatic brain injury, usually mild brain injury, and when both syndromes coexist at times, complicated by a movement disorder.

Patient: As used herein, the term “patient” refers to any organism to which a provided composition is or may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. In some embodiments, a patient is suffering from or susceptible to one or more disorders or conditions. In some embodiments, a patient displays one or more symptoms of a disorder or condition. In some embodiments, a patient has been diagnosed with one or more disorders or conditions. In some embodiments, the patient is receiving or has received certain therapy to diagnose and/or to treat a disease, disorder, or condition.

Pharmaceutical composition: As used herein, the term “pharmaceutical composition” refers to a composition in which an active agent is formulated together with one or more pharmaceutically acceptable carriers. In some embodiments, the active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the term “pharmaceutically acceptable” applied to the carrier, diluent, or excipient used to formulate a composition as disclosed herein means that the carrier, diluent, or excipient must be compatible with the other ingredients of the composition and not deleterious to the recipient thereof.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Prevention: The term “prevention”, as used herein, refers to a delay of onset, and/or reduction in frequency and/or severity of one or more symptoms of a particular disease, disorder or condition. In some embodiments, prevention is assessed on a population basis such that an agent is considered to “prevent” a particular disease, disorder or condition if a statistically significant decrease in the development, frequency, and/or intensity of one or more symptoms of the disease, disorder or condition is observed in a population susceptible to the disease, disorder, or condition. Prevention may be considered complete when onset of a disease, disorder or condition has been delayed for a predefined period of time.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Sample: As used herein, the term “sample” typically refers to a biological sample obtained or derived from a source of interest, as described herein. In some embodiments, a source of interest comprises an organism, such as a microbe, a plant, an animal or a human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

Subject: As used herein “subject” means an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Symptoms are reduced: According to the present invention, “symptoms are reduced” when one or more symptoms of a particular disease, disorder or condition is reduced in magnitude (e.g., intensity, severity, etc.) and/or frequency. For purposes of clarity, a delay in the onset of a particular symptom is considered one form of reducing the frequency of that symptom.

Therapeutic agent: As used herein, the phrase “therapeutic agent” in general refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, a “therapeutic agent” is an agent that has been or is required to be approved by a government agency before it can be marketed for administration to humans. In some embodiments, a “therapeutic agent” is an agent for which a medical prescription is required for administration to humans.

Therapeutically effective amount: As used herein, “therapeutically effective amount” is meant an amount that produces the desired effect for which it is administered. In some embodiments, the term refers to an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a dosing regimen.

Traumatic Brain Injury (TBI): As used herein, the term “traumatic brain injury” is brain damage that results from external forces. In some embodiments, traumatic brain injuries can involve contusion, brain laceration, intracranial hematoma, contrecoup injury, shearing of nerve fibers, intracranial hypertension, hypoxia, hydrocephalus, and subarachnoid hemorrhage. Although the types of external forces resulting in traumatic brain injury are diverse, the most common causes are car accidents, falls, and being struck by or against something (Centers for Disease Control and Prevention, TBI: Get the Facts, 2016). In some embodiments, traumatic brain injury is a physiological disruption of brain function as a result of an external force that is indicated by new onset or worsening of at least one of the following clinical signs, immediately following the event: any period of loss of or a decreased level of consciousness; any loss of memory for events immediately before or after the injury; any alternation in mental state at the time of the injury (confusion, disorientation, slowed thinking, etc.); neurological deficits (weakness, loss of balance, change in vision, praxis, paresis/plegia, sensory loss, aphasia, etc.) that may or may not be transient; intracranial lesion. In some embodiments, external forces may include any of the following events: the head being struck by an object, the head striking an object, the brain undergoing an acceleration/deceleration movement without direct external trauma to the head, a foreign body penetrating the brain, forces generated from events such as a blast or explosion, or other force yet to be defined. In some embodiments, TBI includes instances where the onset of symptoms is not present immediately after the time of injury. In some embodiments, the symptoms may be delayed for days or weeks before they appear clinically. In some embodiments, the severity of TBI can be described as mild, moderate, or severe. In some embodiments, mild traumatic brain injury is defined as: (i) an external injury to the brain; (ii) confusion, disorientation, or loss of consciousness for 30 minutes or less; (iii) Glasgow Coma Scale score of 13 to 15; and (iv) post-traumatic amnesia for less than 24 hours (Bryant, R. Post-traumatic stress disorder vs traumatic brain injury. Dialogues Clin Neurosci, 2011, 13 (3), 251-262). Mill and CAT scans are often normal in mild TBI subjects, but a subject can experience cognitive problems such as headache, difficulty thinking, memory problems, attention deficits, mood swings and frustration. In some embodiments, mild TBI is referred to as a concussion, minor head trauma, minor TBI, minor brain injury, and/or minor head injury. In some embodiments, moderate TBI can involve loss of consciousness between 30 minutes and 24 hours, Glasgow Coma Scale score of 9 to 12, and post-traumatic amnesia between 1 and 7 days. In some embodiments, severe TBI can involve more extended loss of consciousness, usually above 6 hours, post-traumatic amnesia, which typically results in more severe cognitive impairment, and Glasgow Coma Scale of 3 to 8. The International Statistical Classification of Diseases and Related Health Problems, 10th Revision (ICD-10) is an international standard for reporting clinical diagnoses developed by the World Health Organization. In the ICD-10 TBI is classified under section XIX Injury, poisoning and certain other consequences of external causes; S00-S09 Injuries to the head; S06 Intracranial injury. In some embodiments, TBIs can be further assigned classified as belonging to any of S06.0 to S06.9 based on the details of the injury. In some embodiments, TBI includes Chronic Traumatic Encephalopathy (CTE), which is a progressive degenerative disease that affects the brain of subjects that have suffered repeated concussions and TBIs. In some embodiments, CTE is at risk of developing in contact sport athletes, military veterans, and/or civilians exposed to repetitive TBIs. In some embodiments, CTE results in progressive degeneration of brain tissues and the accumulation of Tau proteins. Those skilled in the art will appreciate that Tau proteins initially appear around the blood vessels in the depths of the sulci, but as the disease progresses Tau spreads to other areas of the brain. In some embodiments, a clinical presentation of CTE may include one or more symptoms of memory loss, confusion, impaired judgment, impulse control problems, aggression, depression, anxiety, suicidality, parkinsonism, and/or, eventually, progressive dementia. One of skill in the art will appreciate that disease progression and epidemiology of CTE is largely unknown and is typically verified by post-mortem analysis, however the use of PET to measure Tau protein accumulation in the brain is still being researched and validated. In some embodiments, CTE can be verified in accordance with the present disclosure by analyzing clinical course, which may, for example be indicative of a progressive degenerative process at the cellular level leading to the production of abnormal proteins and progressive loss of brain function. Prior to the present disclosure, agents inducing an amelioration of this process if administered at the time of higher risk, when a second exposure brain injury occurs, have not been available.

Ursodeoxycholic Acid (UDCA) therapy: As used herein, the term “Ursodeoxycholic Acid (UDCA) therapy” refers to administration of UDCA or an analogue, conjugate, derivative, or formulation thereof. In some embodiments, UDCA is in a form selected from the group consisting tauroursodeoxycholic acid, glycoursodexoycholic acid, tauroursodeoxycholate, glycoursodeoxycholate, nortauroursodeoxycholic acid, norglycoursodexoycholic acid, norursodexoycholic acid, and combinations thereof. In some embodiments, UDCA is in a form selected from the group consisting of a bile salt, a carboxylate ion, an amino acid derivative, and combinations thereof. In some embodiments, a UDCA therapy comprises a pharmaceutically acceptable salt.

### DETAILED DESCRIPTION OF CERTAIN EMBODIMENTS

Ursodeoxycholic Acid (UDCA, Ursodiol) has been approved by the United States Food and Drug Association (FDA) for the treatment of primary biliary cirrhosis. The present disclosure provides insight that UDCA is useful in contexts not previously appreciated. For example, the present disclosure provides the insight that UDCA is useful in the treatment of brain disorders, neurodevelopmental disorders, and/or neuropsychiatric disorders. In light of insight provided herein, retrospective analysis of existing data confirm that UDCA therapy will be effective in subjects with brain disorders, neurodevelopmental disorders, and/or neuropsychiatric disorders. Absent the teachings of the present specification one of skill in the art would not have come to such findings.

### Indications

The present disclosure provides use of UDCA in the treatment of brain disorders, neurodevelopmental disorders, and neuropsychiatric disorders. In some embodiments, these disorders present with behavioral abnormalities in the patient, and often are associated with neuroinflammation, oxidative stress, and mitochondrial dysfunction. In some embodiments, the brain disorders, neurodevelopmental disorders, and neuropsychiatric disorders are Autism Spectrum Disorder, Schizophrenia, Rett syndrome, Down Syndrome, Tourette Syndrome, Traumatic Brain Injury, Communication, Speech, and Language Disorders, Mendelsohnn's Syndrome, Fetal Alcohol Spectrum Disorder, Fragile-X syndrome, Attention Deficit Disorder, Angelman Syndrome, Bipolar Disorder, Cerebral Palsy, Landau-Kleffner Syndrome, Phenylketonuria, Prader-Willi Syndrome, Seizure Disorder, or Williams Syndrome.

Autism Spectrum Disorders (ASD) is a broad term that encompasses a group of complex neurodevelopmental disorders including: classical (now regressive) autism; childhood disintegrative disorder; pervasive developmental disorder-not otherwise specified; Asperger syndrome; and more. ASD is characterized by difficulties with social interaction, impaired communication, delayed and atypical language development, repetitive and stereotypic behaviors, obsessional and compulsive behavior and fascinations with part objects, and a lack of social and emotional reciprocity. Such manifestations are found in isolation or in different combinations in most developmental disabilities characterized by cognitive delay.

The onset of ASD is generally before the age of 3 years, and an ASD is usually first diagnosed in early childhood. ASD can range from a severe form, called autistic disorder, through pervasive development disorder not otherwise specified (PDD-NOS), to a much milder form, commonly known as Asperger's syndrome. ASD have a prevalence of 0.6% in the population, affecting many more boys than girls (see, Bertrand et al., Pediatrics 2001; 108:1155-61, Yeargin et al., JAMA 2003; 289:49-55, and Newschaffer et al., Pediatrics 2005; 115:e277-82). Twin and family studies have estimated the heritability of autism as being up to 90%, making it one of the most heritable complex disorders. Rare genetic syndromes and known chromosomal anomalies explain roughly 10% of cases of autism, including Fragile X, tuberous sclerosis, Smith-Lemli-Opitz syndrome, and maternally-inherited duplications of the Prader-Willi/Angelman syndrome region (15q11-13). However, despite high heritability, genetic studies to date have not provided substantial insight into the 90% of autism spectrum disorders with idiopathic etiology.

Childhood disintegrative disorder (CDD), also known as Heller syndrome, is a condition in which children develop normally until age 2-4 years (i.e. later than in Autism and Rett syndrome), but then demonstrate a severe loss of social, communication and other skills. Childhood disintegrative disorder is very much like autism and both involve normal development followed by significant loss of language, social play and motor skills. However, childhood disintegrative disorder typically occurs later than autism, involves a more dramatic loss of skills and is far less common.

Pervasive Developmental Disorder—Not Otherwise Specified (PDD-NOS) is an ASD that describes patients exhibiting some, but not all, of the symptoms associated with other well defined ASDs. Criteria for diagnosis of an ASD include difficulty socializing with others, repetitive behaviors, and heightened sensitivities to certain stimuli. These are all found in the ASDs described above. However, autism, Asperger syndrome, Rett syndrome and childhood disintegrative disorder all have other features that enable their specific diagnosis. When specific diagnosis of one of these four disorders cannot be made, but ASD is apparent, a diagnosis of PDD-NOS is made. Such a diagnosis may result from symptoms starting at a later age than is applicable for other conditions in the spectrum.

Rett Syndrome (RTT) is a neurodevelopmental disorder that almost exclusively affects females (1 in 10:000 live births). RTT is classified as an autism spectrum disorder (Diagnostic and Statistical Manual of Mental Disorders, Fourth Edition—Revised (DSM-IV-R). For a diagnosis of Rett syndrome, the following symptoms are characteristic: impaired development from age 6-18 months; slowing of the rate of head growth starting from between age 3 months and 4 years; severely impaired language; repetitive and stereotypic hand movements; and gait abnormalities (e.g., toe-walking or unsteady stiff-legged walk). There are a number of supportive criteria that may help diagnosis of Rett Syndrome, but are not essential for a diagnosis. These include breathing difficulties, EEG abnormalities, seizures, muscle rigidity and spasticity, scoliosis (curving of the spine), teeth-grinding, small hands and feet in relation to height, growth retardation, decreased body fat and muscle mass, abnormal sleep patterns, irritability or agitation, chewing and/or swallowing difficulties, poor circulation and constipation.

Current therapeutic strategies for the diverse forms of autistic syndromes (e.g., ASD) are mainly targeted to the correction of the most severe behavioral problems or symptoms associated with the main disorder. A number of psychopharmacological agents have been utilized in children with autism, but only to address specific psychiatric or behavioral symptoms, and with mixed results. These agents include: serotonin-related drugs, dopamine-related agents, epinephrine and norepinephrine-related compounds, and a variety of other agents such as opiate antagonists, ACTH, clozapine, risperidone, vitamins B6 and B12 and melatonin. Thus, while children with ASD are prescribed a number of drugs, there is still no accepted rational therapeutic paradigm designed to address autism or its causes.

In some embodiments, an autism spectrum disorder is autistic disorder, asperger's syndrome, aervasive development disorder, Rett syndrome, or childhood disintegrative disorder.

Studies suggest that the prevalence of ASD has increased over the last decade to 1 in 68 children (CDC, 2015), although the cause of this increase is unclear. No treatments currently exist for the core symptoms of ASD, and new and innovative ASD treatment proposals are needed.

There is a growing amount of evidence that individuals with ASD experience high levels of oxidative stress. Levels of the major cellular antioxidant glutathione (GSH) are lower in individuals with ASD (generally 20 to 40%) compared to age-matched controls (Kern et al, 2011). Glutathione is primarily synthesized in the liver from methionine via a transsulfuration reaction. James (2015) found abnormal methionine transsulfuration metabolite levels in autistic children and concluded that these findings reflected a significant decrease in antioxidant capacity.

A broad review of oxidative stress in autism is provided by Chauhan & Chauhan (2006). Levels of pro-oxidants are increased in autism (nitric oxide; xanthine synthase; homocysteine; Hg; Pb; thalidomide; valproic acid; retinoic acid, and more), while levels of antioxidants and antioxidant enzymes are decreased (glutathione; ceruloplasmin; transferrin; superoxide dismutase; glutathione peroxidase; and catalase). As well, the authors state that the repeated finding of increased lipid peroxidation in ASD confirms the hypothesis of increased oxidative stress in autism.

A relationship between autism and inflammatory processes has emerged in the research literature. Abdallah (et al, 2012) found that children developing ASD later in life were more likely to have decreased neonatal levels of Th-1 and Th-2 like cytokines, as wells as, elevated levels of neonatal levels of IL-8 and sIL-bra. As well, marked microglial and astroglial activation in the brains of autistic patients, which induces pro-inflammatory cytokine production, demonstrates the presence of neuroinflammation in autism (Vargas et al, 2005).

Xu (et al, 2015) reviews the research to date regarding the various inflammatory cytokines shown to play a role in autism. The authors summarize that cytokine levels in the blood, brain, and cerebrospinal fluid of autistic subjects differ from those of healthy individuals. A list of cytokines investigated for their involvement with ASD includes: interleukin (IL)-1; IL-1R; IL-1β; IL-2; IL-2R; IL-6; monocyte chemoattractant protein-1; osteopontin; tumor necrosis factor-α; interferon-γ; transforming growth factor-β1; epidermal growth factor; brain-derived neurotrophic factor; and granulocyte-macrophage colony-stimulating factor.

A link has also been established between ASD and autoimmunity. Comi (et al, 1999) demonstrated that the frequency of autoimmune disorders, specifically type 1 diabetes, rheumatoid arthritis, hypothyroidism, and systemic lupus erythematosus, was higher in families with autism. The researchers also found an unusually high prevalence of asphyxia, prematurity, and seizures in the newborn, as well as, perinatal inflammatory disorders (such as, urinary tract, respiratory, and vaginal infections) in the mothers of autistic subjects.

Atladottir (et al, 2009) used data from the Danish Newborn Screening Biobank to show that an increased risk of ASD development was associated with a maternal history of rheumatoid arthritis and celiac disease, as well as, a family history of type 1 diabetes.

Similarly, Brimberg (et al, 2013) analyzed the plasma of 2431 mothers of children with ASD, the largest cohort reported so far, and found elevated levels of anti-brain antibodies. As well, the authors bolstered the conclusions of previous researchers by finding an increased prevalence of autoimmune diseases in the mothers, especially rheumatoid arthritis and systemic lupus erythematosus.

Disturbances of energy metabolism and mitochondrial dysfunction in the brains of autistic patients have been described (Lombard, 1998; Chauhan & Chauhan, 2006). Elevated plasma lactate levels are indicative of mitochondrial dysfunction and are found in approximately 1 in 5 ASD patients (Oliveira, 2005; Correia et al, 2006). Similarly, elevated pyruvate, carnitine, ubiquinone, and alanine levels are found in ASD and imply the presence of mitochondrial dysfunction (Rossignol & Frye, 2012; Frye, 2012). Impaired and/or irregular activity of a variety of mitochondrial metabolic enzymes is documented in ASD, including: NADH oxidase; succinate oxidase; electron transport chain complexes 1-4; ATPase; and more (Ji et al, 2009; Dhillion et al, 2011).

During oxidative metabolism reactive oxygen (ROS) and nitrogen (RNS) species are produced by the mitochondria. The reactive species include superoxide, hydrogen peroxide, and nitric oxide (Giulivi et al, 1998). Damaged mitochondria produce more oxidants than normal functioning mitochondria (Chauhan & Chauhan, 2006).

Mitochondria are also intrinsically involved in certain apoptosis pathways that are triggered by intracellular stresses and lead to membrane permeabilization, apoptotic protein release, and disruption of the mitochondrial membrane potential (Amaral et al, 2009). The membrane permeabilization may occur via the opening of the permeability transition core complex, which is promoted by ROS generation. The excessive ROS generation may also uncouple and inhibit electron transfer in the mitochondria (Kroemer et al, 2007). Apoptosis is associated with ASD (Wie et al, 2014) and gastrointestinal disorders, which frequently occur concomitantly with ASD.

The above discussion has focused mainly on ASD, however oxidative stress, inflammation, and mitochondrial dysfunctions are present in many other neurodevelopmental disorders. For example, Hayashi (2009) found evidence of oxidative stress in Xeroderma pigmentosum, Cockayne's syndrome, spinal muscular atrophy, subacute sclerosing panencephalitis, and progressive myoclonic epilepsy. As well, oxidative stress was found in Down syndrome (Nunomura et al, 2000), Rett syndrome (De Felice et al, 2009), schizophrenia (Prabakaran et al, 2004), seizure disorders (Uttara, 2009), and more.

Inflammation has been found and studied in cerebral palsy, schizophrenia, Parkinson's disease, Alzhelmer's disease, and multiple schlerosis (Stolp & Dziegieleweska, 2009). As well, mitochondrial dysfunction is present in Huntington's disease, Friedreich's ataxia, Alzeimer's disease, amyotrophic lateral sclerosis (Beal, 1998), Down syndrome, Fragile X syndrome, Rett syndrome (Valenti et al, 2014), and schizophrenia (Prabakaran et al, 2004).

In some embodiments, neurodevelopmental disorders are intellectual disability (ID), intellectual and developmental disability (IDD), autism, autism spectrum disorders (e.g., Asperger syndrome), fetal alcohol spectrum disorder, motor disorders (e.g., developmental coordination disorder, stereotypic movement disorder, tic disorders), communication, speech and language disorders, genetic disorders (e.g., fragile-X syndrome), down syndrome, attention deficit hyperactivity disorder, Mendelsohnn's syndrome, schizophrenia and schizotypal disorder.

### Ursodeoxycholic Acid (UDCA)

Recently, UDCA (Ursodiol) was explored for its therapeutic effect in Parkinson's disease (PD), amyotrophic lateral sclerosis (ALS), Huntington's disease (HD), and Alzheimer's disease (AD). UDCA is a pleotropic agent with many cellular targets including: ROS production pathways; apoptosis pathways; endoplasmic reticulum stress mechanisms; and many more (Vang et al, 2014). UDCA is a naturally occurring bile acid that represents ˜1 to 5% of human bile acids, and a much larger fraction in bears (Hagey et al, 1993). Administration of exogenous UDCA can enrich the percentage in the bile pool to ˜55 to 60%, however the spillover into the peripheral circulation is variable and depends on the degree of hepatic extraction (Panini et al, 1995). UDCA has been used in traditional Chinese medicines for over 3000 years (Vang et al, 2014).

Researchers found that administration of 10 pM of UDCA was capable of rescuing the lower cellular ATP levels (40% lower than normal) in parkin-deficient neuronal cell culture models of PD, as well as, PINK1 parkin mutations; PINK1 has specific mitochondrial dysfunction in complex 1. Similarly, UDCA rescued cellular ATP levels in LRRK2G2019S mutant fibroblasts (Mortiboys et al, 2013); a model of familial Parkinson's disease with specific impairments in complex 4 and possibly complex 3. A subsequent study found that mitochondrial rescue via 10 nM UDCA in LRRK2G2019S fibroblasts was independent of whether the patient was a manifesting or non-manifesting carrier (Mortiboys et al, 2015). The same study tested the in-vivo effects of UDCA on LRRK2G2019S transgenic flies and concluded that 2.5 μM UDCA profoundly rescued dopaminergic signaling.

Chun and Low (2012) showed that administration of 50-200 μM UDCA to human dopaminergic SH-SY5Y cells dose-dependently prevented sodium nitroprusside-induced cell death, ROS production, RNS production, mitochondrial membrane potential loss, intracellular reduced glutathione depletion, Bcl-2/Bax ratio decrease, and cytochrome c release; findings that support a mitochondrial therapeutic effect. As well, the results suggested that UDCAs anti-apoptotic action was due to regulating phosphatidylinositide 3′-OH kinase (PI3K) -Akt/PKB pathways.

The glycine acid of UDCA, glycoursodeoxycholic acid (GUDCA), was found to decrease mitochondrial dysfunction, caspase-9 activation, matrix metalloproteinase activation, and nitrogen oxide (NO) generation in a cell model of ALS (Vaz et al, 2015). However, these promising results have not yet translated into clinically significant benefits to ALS patients, and the lack of in-human trials prevents UDCA and/or its conjugates from currently being promoted for off-label use in ALS (The ALS-Untangled Group, 2014). Recently, a preliminary in-human trial of TUDCA (1 g twice daily) in 17 ALS patients resulted in significant improvements on the Amyotrophic Lateral Sclerosis Functional Rating Scale Revised, which indicates TUDCA may be efficacious in treating ALS (Elia et al, 2015).

In a cellular model of HD, which used 3-Nitropropionic acid (3-NP) to irreversibly inhibit succinate dehydrogenase and induce apoptotic neuronal cell death, coincubation with the taurine-conjugated UDCA (TUDCA) resulted in ˜80% reduction in apoptosis. TUDCA coincubation also prevented mitochondrial cytochrome c release and mitochondrial membrane alterations (Rodrigues et al, 2000). The authors also suggest that TUDCA inhibits 3-NP apoptosis by directly inhibiting mitochondrial depolarization and outer membrane disruption, while also modulating Bax (Bcl-2-associated X protein) translocation from the cytosol to the mitochondria.

UDCA and TUDCA were shown to have a variety of therapeutic effects in prion disease, including reduced neuronal loss and astrocytosis in mouse models given 0.006 to 0.01% (wt/wt) of UDCA (Cortez et al, 2015).

It is also known that UDCA suppresses the p53 pro-apoptotic protein (Nang et al, 2014), a protein that has been shown to be elevated in the autistic superior frontal and cerebellar cortices by 67.5% and 38%, respectively (Araghi-Niknam & Fatemi, 2003).

UDCA (ursodiol) received approval in 1987 for use in gallstones and primary biliary cirrhosis (PBC) by the United States Food and Drug Administration (FDA). Ursodiol is indicated for the treatment of patients with primary biliary cirrhosis (PBC). The recommended adult dosage is 13 to 15 mg/kg/day administered in two to four divided doses with food. Ursodiol comes in two dosage forms and strength: 1) 250 mg tablet and 2) 500 mg scored tablet. Scored ursodiol 500 mg tablets can be broken in halves to provide recommended dosage.

Contraindications include patients with complete biliary obstruction and known hypersensitivity or intolerance to ursodiol or any of the components of the formulation. Patients with variceal bleeding, hepatic encephalopathy, ascites or in need of an urgent liver transplant, should receive appropriate specific treatment of ursodiol. Liver function tests (γ-GT, alkaline phosphatase, AST, ALT) and bilirubin levels should be monitored every month for three months after start of therapy, and every six months thereafter. This monitoring will allow the early detection of a possible deterioration of the hepatic function. Treatment discontinuation should be considered if the above parameters increase to a level considered clinically significant in patients with stable historical liver function test levels. Caution should be exercised to maintain patient's bile flow.

Common adverse reactions reported with the use of ursodiol during worldwide postmarketing and clinical experience (≥1%) are: abdominal discomfort, abdominal pain, alopecia, diarrhea, nausea, pruritus, and rash. In a randomized, cross-over study in sixty PBC patients, seven patients (11.6%) reported nine adverse reactions: abdominal pain and asthenia (1 patient), nausea (3 patients), dyspepsia (2 patients) and anorexia and esophagitis (1 patient each). One patient on the twice a day regimen (total dose 1000 mg) withdrew due to nausea. All of these nine adverse reactions except esophagitis were observed with the twice a day regimen at a total daily dose of 1000 mg or greater. However, an adverse reaction may occur at any dose.

The adverse reactions listed in Table 1, presented by system organ class in alphabetical order, have been identified during post approval use of ursodiol. Because these reactions are reported voluntarily from a population of uncertain size, it is not always possible to reliably estimate their frequency or establish a causal relationship to drug exposure.

Table 1: Adverse Reactions

TABLE 1
Adverse Reactions

| Disorder | Reaction |
|---|---|
| Gastrointestinal | abdominal discomfort, |
| disorders | abdominal pain, |
| | constipation, diarrhea, |
| | dyspepsia, nausea, |
| | vomiting |
| General disorders | malaise, peripheral |
| and administration | edema, pyrexia |
| site conditions | |
| Hepatobiliary | jaundice (or |
| disorders | aggravation of pre- |
| | existing jaundice) |
| Immune System | Drug hypersensitivity |
| Disorders | to include facial |
| | edema, urticaria, |
| | angioedema and |
| | laryngeal edema |
| Abnormal | ALT increased, |
| Laboratory | AST increased, |
| Tests | blood alkaline |
| | phosphatase increased, |
| | blood bilirubin |
| | increased, |
| | y-GT increased, |
| | hepatic enzyme |
| | increased, |
| | liver function |
| | test abnormal, |
| | transaminases |
| | increased |
| Musculoskeletal | Myalgia |
| and connective | |
| tissue disorders | |
| Nervous system | dizziness, headache |
| disorders | |
| Respiratory, | Cough |
| thoracic and | |
| mediastinal | |
| disorders | |
| Skin and | alopecia, pruritus, |
| subcutaneous | rash |
| tissue disorder | |

Drug interactions include: Bile Acid Sequestering Agents, Aluminum-based Antacids, and drugs that alter the metabolism of lipids or induce cholestasis may interfere with the action of ursodiol. Bile acid sequestering agents may interfere with the action of ursodiol by reducing its absorption. Aluminum-based antacids may interfere with the action of ursodiol by reducing its absorption.

The mechanism by which UDCA improves PBC is thought to involve a decrease in the number of Human Leukocyte Antigen (HLA) class 1 structures on hepatocyte surfaces. HLA class 1 structures display antigens that are recognized by T lymphocytes and results in initiation of the process of hepatocyte lyses (Mikov et al, 2006). This further supports an immunoregulatory role for UDCA.

In some embodiments, the brain disorders, neurodevelopmental disorders, and neuropsychiatric disorders are selected from the group consisting of: Schizophrenia; ASD (including Classical Autism and Asperger syndrome); Rett syndrome; Down Syndrome; Tourette Syndrome; Traumatic Brain Injury; Communication, Speech, and Language Disorders; Mendelsohnn's Syndrome; Fetal Alcohol Spectrum Disorder; Fragile-X syndrome; Attention Deficit Disorder; Angelman Syndrome; Bipolar Disorder; Cerebral Palsy; Landau-Kleffner Syndrome; Phenylketonuria; Prader-Willi Syndrome; Seizure Disorder; and Williams Syndrome.

In some embodiments, the present disclosure provides a method for treating an autism spectrum disorder (ASD) in a subject in need thereof comprising administering an effective amount of a UDCA therapy. The subject can be one diagnosed with an ASD or one suspected by a treating physician of having an ASD. In some embodiments, the subject is one diagnosed with an ASD. In some embodiments, the subject is a human.

### Patient Populations

In some embodiments, UDCA is administered in accordance with the present disclosure to one or more patients (e.g., to a patient population) as described herein.

In some embodiments, a patient population includes one or more subjects (e.g., comprises or consists of subjects) suffering from brain disorders, neurodevelopmental disorders, and neuropsychiatric disorders. In some embodiments, a patient population includes one or more subjects suffering from Autism Spectrum Disorder (ASD).

In some embodiments, a patient population includes one or more subjects (e.g., comprises or consists of subjects) who received previous therapy for treatment of brain disorders, neurodevelopmental disorders, and neuropsychiatric disorders (e.g., ASD). In some embodiments, a patient population includes one or more subjects (e.g., comprises or consists of subjects) who have not received previous therapy for treatment of brain disorders, neurodevelopmental disorders, and neuropsychiatric disorders (e.g., ASD). In some embodiments, a patient population comprises or consists of patients who have not received previous therapy for treatment of brain disorders, neurodevelopmental disorders, and neuropsychiatric disorders (e.g., ASD).

In some embodiments, a patient who received previous therapy may have received previous therapy for anxiety, depression, obsessive-compulsive disorder and/or attention deficit disorder. In some embodiments, a patient who received previous therapy may have received antipsychotics or anticonvulsants. In some embodiments, a patient may have received previous therapy selected from the group comprising: serotonin-related drugs, dopamine-related agents, epinephrine and norepinephrine-related compounds, opiate antagonists, ACTH, clozapine, risperidone, vitamins B6, vitamin B12, melatonin and combinations thereof.

In some embodiments, a patient population includes one or more subjects (e.g., comprises or consists of subjects) who have received and/or are receiving other therapy, e.g., so that UDCA therapy composition is administered in combination with the other therapy.

In some embodiments, the present invention provides a method of preventing or delaying the onset of one or more brain disorders, neurodevelopmental disorders, and/or neuropsychiatric disorders, comprising administering to a patient identified to be in need of prevention, or delaying the onset, of brain disorders, neurodevelopmental disorders, and neuropsychiatric disorders a prophylactically effective amount of UDCA therapy or a pharmaceutically acceptable salt thereof.

In some embodiments, a patient or patient population may not be (e.g., may exclude) a patient with complete biliary obstruction. In some embodiments, a patient or patient population may not be (e.g., may exclude) a patient with hypersensitivity or intolerance to ursodiol.

In some embodiments, a patient or patient population may be monitored to detect potential adverse events (including for example, abdominal discomfort, abdominal pain, alopecia, diarrhea, nausea, pruritus, elevated creatinine, elevated blood glucose, leukopenia, peptic ulcer, rash, skin rash, thrombocytopenia, etc.). In some embodiments, UDCA may be reduced, suspended, and/or terminated for a patient determined to display one or more signs of such an adverse event.

In some embodiments, a patient or patient population may be monitored for parameters such as liver function tests (e.g., γ-GT, alkaline phosphatase, AST, ALT) and bilirubin level. In some embodiments, UDCA may be reduced, suspended, and/or terminated if parameters increase to a level considered clinically significant in patients. In some embodiments, UDCA may be reduced, suspended, and/or terminated if parameters increase to a level considered clinically significant in patients with stable historical liver function test levels. In some embodiments, a patient or patient population bile flow may be monitored.

In some embodiments, the patient or patient population is pregnant. In some embodiments, the patient or patient population is nursing. In some embodiments, the patient or patient population is a child. In some embodiments, the patient or patient population is an adult.

In some embodiments, the present invention provides use of a compound or a pharmaceutically acceptable salt thereof for the manufacture of a medicament useful for treating, preventing, or delaying the onset of brain disorders, neurodevelopmental disorders, and neuropsychiatric disorders. In some embodiments, the patient or patient population is suffering from ASD. In some embodiments, the patient or patient population is suffering from ASD that is resistant to other therapies. In some embodiments, the patient is a pediatric patient suffering from ASD.

In some embodiments, ursodeoxycholic acid (UDCA) therapy may be administered to prevent or delay onset, or minimize impact of CTE. In some embodiments, UCDA therapy may be administered prior to onset of CTE in a subject. In some embodiments, UDCA therapy may be administered to subjects that have experienced multiple concussions in sports events. In some embodiments, UDC; therapy may be administered prior to onset of CTE in subject (e.g., human) during a stage of preadolescent or adolescent brain development), including for example a subject that participates in, or is intending to participate in, sporting events or activities commonly associated with a risk of concussions. In some embodiments, UDCA therapy may be administered to such subjects before the appearance of one or more symptoms of CTE (e.g., as listed herein).

Without wishing to be bound by any particular theory, the present disclosure proposes that, in some embodiments, UDCA therapy may provide mitochondrial protection to subjects. In some embodiments, UDCA therapy as described herein is administered to a subject in need of mitochondrial protection.

### Dosage and Administration

In general, each active agent (UDCA) for use in accordance with the present invention is formulated, dosed, and administered in a therapeutically effective amount using pharmaceutical compositions and dosing regimens that are consistently with good medical practice and appropriate for the relevant agent(s) and subject. Methods of administering a formulation comprising the compositions of this disclosure can be selected from any method known in the art. These methods include, but are not limited to, oral, parenteral, transdermal, intradermal, intramuscular, intraperitoneal, intravenous, subcutaneous, intranasal, colorectal, rectal, intravaginal, intraventricular, intrathecal, and any combination thereof.

In some embodiments, a dosing regimen for a particular active agent may involve intermittent or continuous administration, for example to achieve a particular desired pharmacokinetic profile or other pattern of exposure in one or more tissues or fluids of interest in the subject receiving therapy.

In some embodiments, different agents administered in combination may be administered via different routes of delivery and/or according to different schedules. Alternatively or additionally, in some embodiments, one or more doses of a first active agent is administered substantially simultaneously with, and in some embodiments via a common route and/or as part of a single composition with, one or more other active agents.

Factors to be considered when optimizing routes and/or dosing schedule for a given therapeutic regimen may include, for example, the particular indication being treated, the clinical condition of a subject (e.g., age, overall health, prior therapy received and/or response thereto, etc.) the site of delivery of the agent, the nature of the agent, the mode and/or route of administration of the agent, the presence or absence of combination therapy, and other factors known to medical practitioners. For example, in the treatment of brain disorders, neurodevelopmental disorders, and neuropsychiatric disorders, relevant features of the indication being treated may include, among other things, one or more of brain disorders, neurodevelopmental disorders, and neuropsychiatric disorders type, stage, etc.

In some embodiments, one or more features of a particular pharmaceutical composition and/or of a utilized dosing regimen may be modified over time (e.g., increasing or decreasing amount of active in any individual dose, increasing or decreasing time intervals between doses, etc.), for example in order to optimize a desired therapeutic effect or response.

In general, type, amount, and frequency of dosing of active agents in accordance with the present invention are governed by safety and efficacy requirements that apply when relevant agent(s) is/are administered to a mammal, preferably a human. In general, such features of dosing are selected to provide a particular, and typically detectable, therapeutic response as compared with what is observed absent therapy. In some embodiments, UDCA will be administered continuously.

In context of the present invention, an exemplary desirable therapeutic response may involve, but is not limited to, inhibition of and/or decreased reactive oxygen and/or nitrogen species generation, neuroinflammatory processes, and mitochondrial dysfunction. Such criteria can be readily assessed by any of a variety of immunological, cytological, and other methods that are disclosed in the literature.

In some embodiments, a UDCA therapy regimen comprises a plurality of doses of a UDCA composition. In some embodiments, a UDCA therapy regimen comprises, for example 2, 5, 10, 20, 30, 60, 90, 180, 365 doses or a number of doses between any two of these values and/or comprises a repeated pattern of doses (e.g., at least one cycle of two daily doses, which cycle may be repeated, optionally with a period of alternative administration, or optionally no administration, separating different cycles).

In some embodiments, a UDCA therapy regimen may be administered to a subject or population of patients known to have consumed, or not consumed, some amount of food before, during or after the administration. The terms “before administration” and “after administration” with respect to food intake may refer to a period of time of about 1, 2, 3, 4, 5, 6, 7, 8, 9, 10, 11, 12, 14, 16, 18, 20, 22, 24, 30, 42, or 72 hours, or longer, before or after the administration. In some embodiments, the term “administering . . . with regard to food intake” implies that the subject or population of patients consumes food before the administration (e.g., fed state). In some embodiments, the term “administering . . . with regard to food intake” implies that the subject or population of patients consumes food after the administration. In some embodiments, the term “administering . . . with regard to food intake” implies that the subject or population of patients consumes food during the administration. Alternatively, in some embodiments, the term “administering . . . with regard to food intake” means the subject or population of patients is in a fasted state during administration. In some embodiments, food intake includes high fat foods or a high fat diet. In some embodiments, a UDCA therapy regimen is administered to a subject in a fasted state. In some embodiments, a UDCA therapy regimen is administered to a subject in a fed state.

The recommended adult dosage for treatment of PBC is 13-15 mg/kg/day UDCA administered in two to four divided doses (FDA, 2004), however UDCA has been well tolerated without significant neurotoxic effects up to 50 mg/kg/day in divided doses (Parry et al, 2010).

The recommended UDCA dose for children is 5-30 mg/kg/day divided into two or three doses. Santovena (et al, 2014) created a liquid-oral 1.5% (w/v) UDCA formulation containing glycerol that was suitable for use in infants and children. The formulation was stable across a variety of conditions and the researchers found that they could consistently obtain the declared active pharmaceutical ingredient value by employing Standard Operating Procedures.

The pharmacokinetics of UDCA was summarized well by Buck (2009). After oral delivery, ˜90% of the exogenous UDCA is absorbed via passive diffusion by the proximal jejunum and ileum. The liver extracts UDCA from the portal vein circulation and subsequently UDCA is conjugated with glycine (GUDCA) or taurine (TUDCA). The conjugates are secreted into bile, concentrated in the gallbladder, and moved to the duodenum via the cystic and common ducts.

UDCA is subjected to extensive enterohepatic recycling and oxidization or reduction by gut bacteria to produce 7-keto-lithocholic acid and lithocholic acid, which are eliminated in the feces; only 1% of oral UDCA doses are excreted via the urine (Buck, 2009). The elimination half-life of UDCA in adults is 3 to 6 days, and steady state concentrations are on average reached within 3 weeks of starting therapy. Bile UDCA levels fall to ˜5 to 10% of the average steady state concentrations following discontinuation of therapy (Buck, 2009).

The bioavailability of orally administered UDCA is well known. Willams (et al, 2000) calculated the area under the curve (AUC), Cmax, and Tmax of UDCA 500 mg doses from USA Ursoflak, Canadian Ursofalk, Ursolvan, and Actigall capsules. The mean AUC was 68.99 μmol/1.6 h-1 for the USA tablet, 59.34 μmol/1.6 h-1 for the Canadian tablet, 55.55 μmol/1.6 h-1 for Ursolvan, and 46.44 μmol/1.6 h-1 for Actigall. The mean Cₘₐₓ values were 24.29, 17.85, 16.63, and 13.32 nmol/mL, respectively. The mean Tₘₐₓ values were 1.82, 2.3, 2.79, and 3.39 h, respectively.

Panini (et al, 1995) increased the bioavailability of oral UDCA by complexation with 2-hydroxypropyl-β-cyclodextrin. The researchers found that administration of just 450 mg UDCA resulted in the following measures: Cₘₐₓ (6.1 μg ml-1); tₘₐₓ (83 minutes); and t_{1/2}(abs) (54 minutes). However, following administration of the complex containing 426 mg UDCA the measures were decreased to: Cₘₐₓ (2.9 μg ml-1), tₘₐₓ (63 minutes), and t_{1/2}(abs) (21 minutes).

Ma (et al, 2014) found that preparing UDCA into submicron emulsions (UDSME) was another way to increase the bioavailability of oral UDCA. Rats receiving 20 mg/kg equivalents of UDCA via UDSME had better bioavailability measures compared to rats receiving coarse UDCA suspension. The bioavailability measures for the UDSME and coarse UDCA groups follows: area under the curve₀₋₂₄ (8.346±0.945; 2.564±1.231 [μg·h/mL]); Tₘₐₓ (1.489±0.2385; 2.215±0.2465 [h]); Cₘₐₓ (0.1562±0.0362; 0.0364±0.0239 [μg/mL]).

Bile acids themselves are quite permeable, and they have been shown to increase the bioavailability of other drugs when complexed in a shared formulation. For example, interferon (INF) α was undetectable in the plasma following hydrophilic suppository base administration. However, when incorporated with 4 and 8 mg sodium ursodeoxycholate INF-α plasma concentrations were 70 and 78 IU/ml, respectively (Mikov et al, 2006).

UDCA serum levels peak 30 to 50 minutes after oral administration. UDCA crosses the blood-brain barrier in a dose-dependent manner. In a study of 18 ALS patients a strong statistically significant correlation was discovered between serum UDCA concentrations 1 hour after dosing and cerebrospinal fluid concentrations 2 hours after dosing (Parry et al, 2010).

Transdermal and parenteral administration of UDCA has not been extensively explored in the literature. Most compoundings of UDCA into transdermal formulations are intended for veterinary use. There has been hesitation to deliver UDCA transdermally since its site of action has historically been viewed as the liver, and traditional oral administration provides significant first-pass effects that deliver UDCA directly to the liver (Davidson, 2003). UDCA has a variety of sights of action, including the nervous system, and topical administration can transport UDCA into systemic circulation where UDCA can reach the nervous system.

In some embodiment, UDCA treatment will be administered to patients in a form capable of delivering 1 to 50 mg UDCA (or equivalents) per kg of bodyweight per day. This may require that the treatment be administered in divided doses.

In some embodiments, a UDCA therapy regimen comprises at least one (or includes or consists of exactly one) dose of about 200 mg, 250 mg, 300 mg, 350 mg, 400 mg, 450 mg, 500 mg, 550 mg, 600 mg or a dose between any two of these values, of UDCA or a pharmaceutically acceptable salt thereof.

In some embodiments, a UDCA therapy regimen comprises at least one (or includes or consists of exactly one) dose of a composition that delivers about 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1250 mg, 1500 mg, 1750 mg, 2000 mg or a dose between any two of these values, of UDCA or a pharmaceutically acceptable salt thereof.

In some embodiments, a UDCA therapy regimen comprises at least one (or includes or consists of exactly one) dose of about 10-20 mg/kg/day, 10-15 mg/kg/day, 15-20 mg/kg/day, 10-13 mg/kg/day, 11-20 mg/kg/day, 12-20 mg/kg/day, 13-20 mg/kg/day, 12-16 mg/kg/day, 13-15 mg/kg/day or a dose between any two of these values, of UDCA or a pharmaceutically acceptable salt thereof.

In some embodiments, a UDCA therapy regimen is administered in two to four divided doses with food. In some embodiments, a UDCA therapy regimen comprises 13 to 15 mg/kg/day administered in two to four divided doses with food. In some embodiments, a UDCA therapy regimen is administered to an adult. In some embodiments, a UDCA therapy regimen is administered to a child.

### Formulations

In some embodiments, UDCA therapy, or a formulation comprising UDCA, is used for ameliorating the effects of brain disorders, neurodevelopmental disorders, and neuropsychiatric disorders. In some embodiments, ursodeoxycholic acid (UDCA) therapy is or comprises UDCA or an analog or conjugate or derivative thereof and/or a pharmaceutically acceptable carrier.

In some embodiments, UDCA therapy is present in its conjugate forms; namely its taurine conjugate form, TUDCA, and its glycine conjugate form, GUDCA. In some embodiments, UDCA and its conjugates are present in their carboxylate ion forms or as bile salts. In some embodiments, UDCA and its conjugates are present as amino acid derivatives. In some embodiments, UDCA and its conjugates are present in analog forms (e.g. norUDCA, norTUDCA, and norGUDCA).

In some embodiments, UDCA therapy is in various forms. In some embodiments, UDCA therapy is tauroursodeoxycholic acid, glycoursodexoycholic acid, tauroursodeoxycholate, glycoursodeoxycholate, nortauroursodeoxycholic acid, norglycoursodexoycholic acid or norursodexoycholic acid. In some embodiments, UDCA is a combination of forms. For example, UDCA therapy is a combination of one or more of tauroursodeoxycholic acid, glycoursodexoycholic acid, tauroursodeoxycholate, glycoursodeoxycholate, nortauroursodeoxycholic acid, norglycoursodexoycholic acid and/or norursodexoycholic acid.

In some embodiments, UDCA therapy is present in any variety of combinations with its aforementioned conjugates, derivatives, and analogs. In some embodiments, a UDCA therapy formulation may include cyclodextrin(s) or submicron emulsions, which increase the bioavailability. In some embodiments, a UDCA therapy formulation comprises or consists of submicron or nanoemulsions. In some embodiments, submicron or nanoemulsions comprise or consist of ursodeoxycholic acid or an analog or conjugate or derivative thereof.

In some embodiments, the present disclosure encompasses UDCA therapy formulated as a cream, gel, liquid, intramuscular formulation, or transdermal patch. In some embodiments, UDCA therapy comprises or consists of UDCA and a pharmaceutically acceptable carrier. In some embodiments, the pharmaceutically acceptable carrier may contain: olive oil, tea tree oil, carbomers, alcohol, water, trolamine, polyethylene glycol, cera alba, water, lecithin, rutin, sodium hydroxide, sorbitan olivate, cetearyl olivate, cetyl palmitate, sorbitan palmitate, potassium citrate, ethoxydiglycol, caprylyl glycol, malaleuca alternifolia lead oil, inulin lauryl carbamate, dehydroacetic acid, benzoic acid, phenoxyethanol, sodium benzoate, potassium sorbate, calendula officinalis flower extract, glycerin, peucedanum ostruthium leaf extract, nymphacea cearulea flower extract, sodium phytate, xanthan gum, isopropyl palmitate, argania spinosa kernel oil, ribes nigrum seed oil, rubus idaeus seed oil, and dromiceius. In some embodiments, PCCA Lipoderm Base is an appropriate pharmaceutically acceptable carrier.

In some embodiments, UDCA therapy is in the form of the bile salt, carboxylate ion, amino acid derivative, and combinations thereof. In some embodiments, UDCA therapy is in the form of the bile salt, carboxylate ion, amino acid derivative, and combinations thereof of the forms selected from the group consisting of tauroursodeoxycholic acid, glycoursodexoycholic acid, tauroursodeoxycholate, glycoursodeoxycholate, nortauroursodeoxycholic acid, norglycoursodexoycholic acid, norursodexoycholic acid, and combinations thereof.

In some embodiments, UDCA therapy comprises a pharmaceutically acceptable carrier which consists or comprises of components selected from the group consisting of olive oil, tea tree oil, carbomer, isopropyl alcohol, purified water and/or trolamine. In some embodiments, the components are in approximate and relative amounts, for example: olive oil in the amount of about 4 to 5 mL, tea tree oil in the amount of about 0.01 to 1.0 mL, carbomer in the amount of about 0.1 to 1 g, isopropyl alcohol in the amount of about 20 to 40 mL, purified water in the amount of about 5 to 15 mL; and trolamine in the amount of about 0.1 to 1 ml. In some embodiments, the components are in approximate and relative amounts, for example: olive oil in the amount of 4.9 ml, tea tree oil in the amount of 0.1 ml, carbomer in the amount of 0.225 g, isopropyl alcohol in the amount of 32.14 ml, purified water in the amount of 12.5 ml, and trolamine in the amount of 0.3 ml.

In some embodiments, UDCA therapy comprises a pharmaceutically acceptable carrier which consists or comprises of cera alba, water, lecithin, rutin, sodium hydroxide, sorbitan olivate, cetearyl olivate, cetyl palmitate, sorbitan palmitate, potassium citrate, ethoxydiglycol, caprylyl glycol, malaleuca alternifolia lead oil, inulin lauryl carbamate, dehydroacetic acid, benzoic acid, phenoxyethanol, sodium benzoate, potassium sorbate, calendula officinalis flower extract, glycerin, peucedanum ostruthium leaf extract, nymphacea cearulea flower extract, sodium phytate, xanthan gum, isopropyl palmitate, argania spinosa kernel oil, ribes nigrum seed oil, rubus idaeus seed oil, dromiceius and any combination thereof. In some embodiments, the components are in approximate and relative amounts, for example: cera alba in the amount of about 1 to 3%, water, lecithin in the amount of about 1 to 4%, rutin, sodium hydroxide, sorbitan olivate in the amount of about 3 to 6%, cetearyl olivate, cetyl palmitate, sorbitan palmitate in the amount of about 2 to 4%, potassium citrate in the amount of 0.1 to 1.0%, ethoxydiglycol in the amount of about 1 to 3%, caprylyl glycol in the amount of about 1 to 2%, malaleuca alternifolia lead oil in the amount of about 0.01 to 0.5%, inulin lauryl carbamate in the amount of about 0.1 to 1.0%, dehydroacetic acid in the amount of about 0.5 to 2.00%, benzoic acid, phenoxyethanol, sodium benzoate in the amount of about 1 to 3%, potassium sorbate, calendula officinalis flower extract, glycerin, peucedanum ostruthium leaf extract in the amount of about 0.5 to 2%, nymphacea cearulea flower extract in the amount of about 1 to 4%, sodium phytate in the amount of about 0.01 to 1.0%, xanthan gum in the amount of about 0.1 to 1.0%, isopropyl palmitate in the amount of about 3 to 6%, argania spinosa kernel oil in the amount of about 0.5 to 2%, ribes nigrum seed oil in the amount of about 2 to 4%, rubus idaeus seed oil in the amount of about 1 to 2%, and dromiceius in the amount of about 3 to10%. In some embodiments, the components are in approximate and relative amounts, for example: cera alba in the amount of 2.50%, water, lecithin in the amount of 2.00%, rutin, sodium hydroxide, sorbitan olivate in the amount of 4.00%, cetearyl olivate, cetyl palmitate, sorbitan palmitate in the amount of 3.00%, potassium citrate in the amount of 0.60%, ethoxydiglycol in the amount of 1.50%, caprylyl glycol in the amount of 0.80%, malaleuca alternifolia lead oil in the amount of 0.10%, inulin lauryl carbamate in the amount of 0.55%, dehydroacetic acid in the amount of 1.00%, benzoic acid, phenoxyethanol, sodium benzoate in the amount of 2.00%, potassium sorbate, calendula officinalis flower extract, glycerin, peucedanum ostruthium leaf extract in the amount of 1.00%, nymphacea cearulea flower extract in the amount of 2.00%, sodium phytate in the amount of 0.05%, xanthan gum in the amount of 0.40%, isopropyl palmitate in the amount of 4.50%, argania spinosa kernel oil in the amount of 1.50%, ribes nigrum seed oil in the amount of 3.00%, rubus idaeus seed oil in the amount of 2.00%, and dromiceius in the amount of 5.00%.

### Combination Therapy

In some embodiments, UDCA therapy is utilized in combination with one or more other therapeutic agents or modalities. In some embodiments the combination shows a synergistic effect in treating brain disorders, neurodevelopmental disorders, and neuropsychiatric disorders. Combination therapy refers to any form of administration combining two or more different therapeutic compounds such that the second compound is administered while the previously administered therapeutic compound is still effective in the body. The different therapeutic compositions can be administered either in the same formulation or in a separate formulation, either simultaneously or sequentially.

Known compounds or treatments that show therapeutic efficacy in treating brain disorders, neurodevelopmental disorders, or neuropsychiatric disorders may include, for example, one or more of isoflavonoids, N-methyl-D-aspartate receptor antagonists, anticholinergics, valproic acid, and combinations thereof.

In some embodiments, UDCA (and/or other therapy with which it is combined) may be combined with one or more palliative (e.g., pain relieving, anti-nausea, anti-emesis, etc.) therapies, particularly when it relieves one or more symptoms known to be associated with the relevant disease, or with another disease, disorder or condition to which a particular subject is susceptible or from which the particular disease patient is suffering.

In some particular embodiments, UDCA therapy as described herein is administered in combination with one or more approved therapies for treatment of an autism spectrum disorders. In some embodiments, UDCA therapy may be combined with therapies used to treat anxiety, depression, obsessive-compulsive disorder and/or attention deficit disorder. In some embodiments, UDCA therapy may be combined with antipsychotics or anticonvulsants. In some embodiments, UDCA therapy may be combined with serotonin-related drugs, dopamine-related agents, epinephrine and norepinephrine-related compounds, opiate antagonists, ACTH, clozapine, risperidone, vitamins B6, vitamin B12, melatonin and combinations thereof.

In some embodiments, UDCA therapy is administered with one or more therapeutic agents useful for treating an ASD. Such therapeutic agents include agents for treating any of the symptoms of ASD. In some embodiments, a therapeutic agent useful for treating an ASD is a selective serotonin reuptake inhibitor, a typical antipsychotic, an atypical antipsychotic, a serotonin-norepinephrine reuptake inhibitor, a stimulant, a dopamine receptor agonist, secretin or oxytocin. Examples of such therapeutic agents useful in the provided methods include selective serotonin reuptake inhibitors (such as Citalopram, Dapoxetine, Escitalopram, Fluoxetine, Fluvoxamine, Indalpine, Paroxetine, Sertraline, Vilazodone and Zimelidine), typical antipsychotics (such as Chlorpromazine, Thioridazine, Mesoridazine, Levomepromazine, Loxapine, Molindone, Perphenazine, Thiothixene, Trifluoperazine, Haloperidol, Fluphenazine, Droperidol, Zuclopenthixol, Flupentixol and Prochlorperazine), atypical antipsychotics (such as Amisulpride, Aripiprazole, Asenapine, Blonanserin, Clotiapine, Clozapine, Iloperidone, Llurasidone, Mosapramine, Olanzapine, Paliperidone, Perospirone, Quetiapine, Remoxipride, Risperidone, Sertindole, Sulpiride, Ziprasidone, Zotepine, Bifeprunox, Pimavanserin and Vabicaserin), serotonin-norepinephrine reuptake inhibitors (such as venlafaxine and duloxetine), anticonvulsants (such as Tegretol, Lamictal, Topamax or Depakote), stimulants (such as Adderall, Concerta, Dexedrine, Focalin, Metadate, Methylin, Ritalin and Vyvanse), dopamine receptor agonists (such as Aplindore, Apomorphine, Bromocriptine, Cabergoline, Ciladopa, Dihydroergocryptine, Lisuride, Pardoprunox, Pergolide, Piribedil, Pramipexole, Ropinirole and Rotigotine), secretin and oxytocin.

In some embodiments, an UDCA therapy is administered in conjunction with a behavioral-based, psychosocial-based, or psychological-based therapy for treatment of an ASD. Intensive, sustained special education programs and behavioral therapy are commonly utilized to help individuals diagnosed with or suspected of having an ASD acquire self-care, social and job skills, improved functioning, and decreased symptom severity and maladaptive behavior. Examples of such behavioral-based, psychosocial-based, or psychological-based therapy for treatment of an ASD useful in the provided methods include behavior analysis, early intensive behavior intervention (EIBI; a.k.a. the LOVAAS method), pivotal response therapy, aversion therapy, the social communication, emotional regulation, transactional support (SCERTS) model, relationship development intervention, sensory integration, massage therapy, animal-assisted therapy, neurofeedback, patterning, packing, developmental model-based therapy, structured teaching, speech and language therapy, social skills therapy, and occupational therapy.

In some embodiments, agents used in combination are administered according to a dosing regimen for which they are approved for individual use. In some embodiments, however, combination with UDCA permits another agent to be administered according to a dosing regimen that involves one or more lower and/or less frequent doses, and/or a reduced number of cycles as compared with that utilized when the agent is administered without UDCA. Alternatively or additionally, in some embodiments, an appropriate dosing regimen involves higher and/or more frequent doses, and/or an increased number of cycles as compared with that utilized when the agent is administered without UDCA.

In some embodiments, one or more doses of agents administered in combination are administered at the same time; in some such embodiments, agents may be administered in the same composition. More commonly, however, agents are administered in different compositions and/or at different times. In some embodiments, UDCA is administered sequentially and/or concurrently with other therapeutic agents.

### EXAMPLES

### Example 1: ASD Patients Treated with UDCA

UDCA may be administered to a patient or patient population determined to have ASD. For example, UDCA therapy may be administered to patients who meet the current Diagnostic and Statistical Manual for Mental Disorders (DSM-IV-TR) for Autism (Autistic Disorder), screened by the Social Communication Questionnaire and confirmed by the Autism Diagnostic Interview-Revised. Patients may be randomly assigned to receive either active treatment (UDCA) or placebo for 12 weeks. Primary outcome measures to determine efficacy of treatment with UDCA versus placebo may include changes in the Aberrant Behavior Checklist (ABC) between baseline and Week 12/Termination visit. Secondary outcome measures to determine efficacy of treatment with UDCA versus placebo may include changes in the Social Responsiveness Scale and various ABC subscales between baseline and Week 12/Termination visit. A Phase-II trial would entail enrolling approximately 60 patients to determine efficacy with a Phase-III trial to follow that would enroll approximately 300 patients.

### REFERENCES

Abdallah, M., Larsen, N., Mortensen, E., Atladottir, H., Norgaard-Pedersen, B., Bonefeld-Jorgensen, E., et al. (2012). Neonatal levels of cytokines and risk of autism spectrum disorders: An exploratory register-based historic birth cohort study utilizing the Danish Newborn Screening Biobank. J. Neuroimmunol.
Amaral, J., Viana, R., Ramalho, R., Steer, C., & Rodrigues, C. (2009). Bile acids: regulation of apoptosis by ursodeoxycholic acid. Journal of Lipid Researh, 50, 1721-1734.
Araghi-Niknam, M., & Fatemi, S. (2003). Levels of Bcl-2 and P53 are altered in superior frontal and cerebellar cortices of autistic subjects. Cell Mol Neurobiol, 23(6), 945-952.
Atladottir, H., Pederson, M., Thorsen, P., Mortensen, P., Deleuran, B., Eaton, W., et al. (2009). Association of family history of autoimmune diseases and autism spectrum disorders. Pediatrics, 124(2), 687-694.
Beal, M. (1998). Mitochondrial dysfunction in neurodegenerative diseases. Biochimica et Biophysica Acta (BBA)—Bioenergetics, 1366(1-2), 211-223.
Brimberg, L., Sadiq, A., Gregersen, P., & Diamond, B. (2013). Brain-reactive IgG correlates with autoimmunity in mothers of a child with an autism spectrum disorder. Mol Psychiatry, 18(11), 1171-1177.
Buck, M. (2009). Use of Ursodiol in Infants and Children. Pediatric Pharmacotherapy, 15(2).
Centers for Disease Control and Prevention. (2015, Aug. 12). Autism Spectrum Disorder (ASD): Data & Statistics. Retrieved Aug. 21, 2015, from Centers for Disease Control and Prevention Website: http://www.cdc.gov/ncbddd/autism/data.html
Centers for Disease Control and Prevention. (2016, Jun. 14). About the CDC-Kaiser ACE Study. Retrieved 9 Sep. 2016, from Injury Prevention & Control: Division of Violence Prevention: https://www.cdc.gov/violenceprevention/acestudy/about.html
Chauhan, A., & Chauhan, V. (2006). Oxidative stress in autism. Pathopysiology, 13, 171-181.
Chun, H., & Low, W. (2012). Ursodeoxycholic acid suppresses mitochondria-dependent programmed cell death induced by sodium nitroprusside in SH-SY5Y cells. Toxicology, 292, 105-112.
Comi, A., Zimmerman, A., Frye, V., Law, P., & Peedon, J. (1999). Familial Clustering of Autoimmune Disorders and Evaluation of Medical Risk Factors in Autism. 14(6), 388-394.
Correia, C., Coutinho, A., Diogo, L., Grazina, M., Marques, C., Miguel, T., et al. (2006). Brief report: High frequency of biochemical markers for mitochondrial dysfunction in autism: no association with the mitochondrial aspartate/glutamate carrier SLC25A12 gene. J Autism Dev Disord., 36(8), 1137-1140.
Cortez, L., Campeau, J., Norman, G., Kalayil, M., Van der Merwe, J., McKenzie, D., et al. (2015). Bile Acids Reduce Prion Conversion, Reduce Neuronal Loss, and Prolong Male Survival in Models of Prion Disease. J. Virol., 89(15), 7660-7672.
Davidson, G. (2003). Veterinary transdermal medications: A-Z. International Journal of Pharmaceutical Compounding, 7(2), 106-113.
De Felice, C., Ciccoli, L., Leoncini, S., Signorini, C., Rossi, M., Vannuccini, L., et al. (2009). Systemic oxidative stress in classic Rett syndrome. Free Radical Biology and Medicine, 47(4), 440-448.
Dhillion, S., Hellings, J., & Butler, M. (2011). Genetics and Mitochondrial Abnormalities in Autism Spectrum Disorders: A Review. Curr Genomics, 12(5), 322-332.
Elia, A., Lalli, S., Monsurro, M., Sagnelli, A., Taiello, A., Reggiori, B., et al. (2015). Tauroursodeoxycholic acid in the treatment of patients with amyotrophic lateral sclerosis. European Journal of Neurology, 0, 1-8.
Food and Drud Administration. (2004, July 21). Retrieved 08 27, 2015, from U.S. Food and Drug Administration: http://www.fda.gov/ohrms/dockets/dockets/05p0315/05p-0315-cp00001-03-Tab-02-voltpdf
Frye, R. (2012). Biomarkers of Abnormal Energy Metabolism in Children with Autism Spectrum Disorder. North American Journal of Medicine and Science, 5(3), 141-147.
Giulivi, C., Poderoso, J., & Boveris. (1998). Production of Nitric Oxide by Mitochondria. Journal of Biological Chemistry, 273(18), 11038-11043.
Hagey, L., Crombie, D., Espinosa, E., Carey, M., Igimi, H., & Hofmann, A. (1993). Ursodeoxycholic acid in the Ursidae: biliary bile acids in bears, pandas, and related carnivores. Journal of Lipid Research, 34, 1911-1917.
Hayashi, M. (2009). Oxidative stress in developmental brain disorders. Neuropathology, 29(1), 1-8.
James, S. (n.d.). Oxidative Stress in Autism—Impaired transsulfuration and oxidative stress in ASD: Improvement with targeted nutrition. Retrieved 08 24, 2015, from Autism Research Institute: http://www.autism.com/pro_research_oxidativestress
Ji, L., Chauhan, A., Brown, W., & Chauhan, V. (2009). Increased activities of Na+/K+−ATPase and Ca2+/Mg2+−ATPase in the frontal cortex and cerebellum of autistic individuals. Life Sciences, 85(23), 788-793.
Kern, J., Geier, D., Adams, J., Garver, C., Audhya, T., & Geier, M. (2011). A clinical trial of glutathione supplementation in autism spectrum disorders. Med Sci Monk., 17(17), 677-682.
Kroemer, G., Galluzi, L., & Brenner, C. (2007). Mitochondrial Membrane Permeabilization in Cell Death. Physiological Reviews, 87(1), 99-163.
Lombard, J. (1998). Autism: a mitochondrial disorder? 50(6), 497-500.
Ma, Y., Li, G., Xu, J., Zhang, J., Zhang, Z., Xiao, H., et al. (2014). Combination of submicroemulsion and phospholipid complex for novel delivery of ursodeoxycholic acid. Pharmaceutical Development and Technology, 19(3), 363-372.
Mikov, M., Fawcett, J., Kuhajda, K., & Kevresan, S. (2006). Pharmacology of Bile Acids and their Derivatives: Absorption Promoters and Therapeutic Agents. European Journal of Drug Metabolism and Pharmacokinetics, 31(3), 237-251.
Mortiboys, H., Aasly, J., & Bandmann, O. (2013, Sep. 2). Ursocholanic acid rescues mitochondrial function in common forms of familial Parkinson's disease. Brain, 1-13.
Nunomura, A., Perry, G., Pappolla, M., Friedland, R., Hirai, K., Chiba, S., et al. (2000). Neuronal oxidative stress precedes amyloid-beta deposition in Down syndrome. J Neuropathol Exp Neurol., 59(11), 1011-1017.
Oliverira, G., Diogo, L., Grazina, M., Garcia, P., Ataide, A., Marques, C., et al. (2005). Mitochondrial dysfunction in autism spectrum disorders: a population-based study. Developmental Medicine & Child Neurology, 47(3), 185-189.
Panini, R., Vandelli, M., Forni, F., Pradelli, J., & Salvioli, G. (1995). Improvement of ursodeoxycholic acid bioavailability by 2-hydroxypropyl-beta-cyclodextrin complexation in healthy volunteers. Pharmacological Research, 31(314).
Parry, G., Rodrigues, C., Aranha, M., Hilbert, S., Davey, C., Kelkar, P., et al. (2010). Safety, Tolerability, and Cerebrospinal Fluid Penetration of Ursodeoxycholic Acid in Patients With Amyotrophic Lateral Sclerosis. Clin Neuropharm, 33, 17-21.
Prabakaran, S., Swatton, J., Ryan, M., Huffaker, S., Huang, J., Griffin, J., et al. (2004). Mitochondrial dysfunction in schizophrenia: evidence for compromised brain metabolism and oxidative stress. Molecular Psychiatry, 9, 684-697.
Rodrigues, C., Stieers, C., Keene, C., Ma, X., Kren, B., Low, W., et al. (2000). Tauroursodeoxycholic acid partially prevents apoptosis induced by 3-nitropropionic acid: evidence for a mitochondrial pathway independent of the permeability transition. J Neurochem, 75(6), 2368-2379.
Rossignol, D., & Frye, R. (2012). Mitochondrial dysfunction in autism spectrum disorders: a systematic review and meta-analysis. Mol Psychiatry, 17(3), 290-314.
Santovena, A., Sanchez-Negrin, E., Charola, L., Llabres, M., & F. J. (2014). Study of quality and stability of ursodeoxycholic acid formulations for oral pediatric administration. International Journal of Pharmaceutics, 477, 32-38.
Stolp, H., & Dziegieleweska, K. (2009). Review: Role of developmental inflammation and blood-brain barrier dysfunction in neurodevelopmental and neurodegenerative diseases. Neuropathology and Applied Neurobiology, 35, 132-146.
The ALS-Untangled Group. (2014). ALSUntangled No. 25: Ursodiol. Amyotrophic Lateral Sclerosis and Frontotemporal Degeneration, 15, 475-478.
Uttara, B., Singh, A., Zamboni, P., & Mahajan, R. (2009). Oxidative Stress and Neurodegenerative Diseases: A Review of Upstream and Downstream Antioxidant Therapeutic Options. Curr Neuropharmacol, 7(1), 65-74.
Valenti, D. d., De Filippis, B., Henrion-Caude, A., St Vacca, R. (2014). Mitochondrial dysfunction as a central actor in intellectual disability-related diseases: An overview of Down syndrome, autism, Fragile X and Rett syndrome. Neuroscience & Biobehavioral Reviews, 46(2), 202-217.
Vang, S., Longley, K., Steer, C., & Low, W. (2014). The Unexpected Uses of Urso- and Tauroursodeoxycholic Acid in the Treatment of Non-liver Diseases. Glob Adv Health Med, 3(3), 58-69.
Vargas, D., Nascimbene, C., Krishnan, C., Zimmerman, A., & Pardo, C. (2005). Neuroglial activation and neuroinflammation in the brain of patients with autism. Annals of Neurology., 57(1), 67-81.
Vaz, A., Cunha, C., Gomes, C., Schmucki, N., Barbosa, M., & Brites, D. (2015). Glycoursodeoxycholic Acid Reduces Matrix Metalloproteinase-9 and Caspase-9 Activation in a Cellular Model of Superoxide Dismutase-1 Neurodegeneration. Nol Neurobiol, 51, 864-877.
Wei, H., Alberts, I., & Li, X. (2014). The apoptotic perspective of autism. Int J Dev Neurosci, 36, 13-18.
Williams, C., Al-Knawy, B., & Blanchard, W. (2000). Bioavailabilty of for ursodeoxycholic acid preparations. Aliment Pharmacol Ther, 14, 1133-1139.
World Health Organization. (n.d.). Adverse Childhood Experiences International Questionnaire (ACE-IQ). Retrieved 9 9, 2016, from Violence and Injury Prevention: http://www.who.int/violence_injury_prevention/violence/activities/advers e_childhood_experiences/en/
Xu, N., Li, X., & Zhong, Y. (2015). Inflammatory Cytokines: Potential Biomarkers of Immunologic Dysfunction in Autism Spectrum Disorders. Mediators of Inflammation, 10.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A method comprising
administering therapy to a subject suffering from or susceptible to a disease, disorder or condition selected from the group consisting of brain disorders, neurodevelopmental disorders, and neuropsychiatric disorders, wherein the therapy comprises administration of ursodeoxycholic acid (UDCA) therapy.

2. The method of claim 2, where the brain disorder, neurodevelopmental disorder, and neuropsychiatric disorder is selected from the group consisting of Autism Spectrum Disorder, Schizophrenia, Rett syndrome, Down Syndrome, Tourette Syndrome, Traumatic Brain Injury, Communication, Speech, and Language Disorders, Mendelsohnn's Syndrome, Fetal Alcohol Spectrum Disorder, Fragile-X syndrome, Attention Deficit Disorder, Angelman Syndrome, Bipolar Disorder, Cerebral Palsy, Landau-Kleffner Syndrome, Phenylketonuria, Prader-Willi Syndrome, Seizure Disorder, and Williams Syndrome.

3. The method of claim 2, wherein the disorder is Autism Spectrum Disorder.

4. The method of claim 1, wherein the UDCA is a form selected from the group consisting of tauroursodeoxycholic acid, glycoursodexoycholic acid, tauroursodeoxycholate, glycoursodeoxycholate, nortauroursodeoxycholic acid, norglycoursodexoycholic acid, norursodexoycholic acid, and combinations thereof.

5. The method of claim 1 or claim 4, wherein the ursodeoxycholic acid (UDCA) is in a form selected from the group consisting of a bile salt, a carboxylate ion, an amino acid derivative, and combinations thereof.

6. The method of claim 1, wherein the UDCA therapy is administered by a route selected from the group consisting of oral, parenteral, transdermal, intradermal, intramuscular, intraperitoneal, intravenous, subcutaneous, intranasal, colorectal, rectal, intravaginal, intraventricular, intrathecal, and combination thereof.

7. A transcutaneous formulation of ursodeoxycholic acid (UDCA) therapy, wherein the formulation comprises ursodeoxycholic acid or an analog or conjugate or derivative thereof; and a pharmaceutically acceptable carrier appropriate for transcutaneous delivery.

8. The formulation of claim 7, wherein the formulation comprises or consists of a cream carrier.

9. The formulation of claim 7, wherein the carrier comprises or consists of polyethylene glycol (PEG).

10. The formulation of claim 7, wherein the formulation is a gel.

11. The formulation of claim 7, wherein the formulation is a liquid.

12. The formulation of claim 7, wherein the carrier comprises or consists of olive oil, tea tree oil, a carbomer, an alcohol, water trolamine and any combination thereof.

13. The formulation of claim 12, wherein the tea tree oil is selected from the group consisting of pamitolic acid, oleic acid, 11-Eicosenoic acid, docosenoic acid, docodiesnoic acid, 9-Gadoelic acid and any combination thereof.

14. The formulation of claim 12, wherein the alcohol is or comprises isopropyl alcohol.

15. The formulation of claim 12, wherein the water is or comprises purified water.

16. The formulation of claim 7, wherein the pharmaceutically acceptable carrier consists or comprises of components selected from the group consisting of olive oil, tea tree oil, carbomer, isopropyl alcohol, purified water and/or trolamine.

17. An intramuscular formulation of ursodeoxycholic acid or an analog or conjugate or derivative thereof, wherein the formulation comprises ursodeoxycholic acid or an analog or conjugate or derivative thereof and a pharmaceutically acceptable carrier appropriate for intramuscular delivery.

18. The formulation of any one of claims 7 and 17, wherein the carrier comprises or consists of cera alba, water, lecithin, rutin, sodium hydroxide, sorbitan olivate, cetearyl olivate, cetyl palmitate, sorbitan palmitate, potassium citrate, ethoxydiglycol, caprylyl glycol, malaleuca alternifolia lead oil, inulin lauryl carbamate, dehydroacetic acid, benzoic acid, phenoxyethanol, sodium benzoate, potassium sorbate, calendula officinalis flower extract, glycerin, peucedanum ostruthium leaf extract, nymphacea cearulea flower extract, sodium phytate, xanthan gum, isopropyl palmitate, argania spinosa kernel oil, ribes nigrum seed oil, rubus idaeus seed oil, dromiceius and any combination thereof.

19. The formulation of any one of the preceding claims, wherein the water is or comprises purified water.

20. The formulation of any one of claims 7 and 17, wherein the pharmaceutically acceptable carrier comprises or consists of PCCA Lipoderm Base.

21. The formulation of any one of claims 7 and 17, wherein the formulation comprises or consists of submicron or nanoemulsions, which submicron or nanoemulsions comprise or consist of ursodeoxycholic acid or an analog or conjugate or derivative thereof.

22. The formulation of any one of claims 7 and 17, wherein the formulation comprises or consists of a cyclodextrin.

23. A method comprising steps of administering to a subject a formulation as set forth in any one of claims 7-22.

24. The method of claim 23, wherein the subject has a disorder selected from a group consisting of brain disorders, neurodevelopmental disorders, neuropsychiatric disorders.

25. The method of claim 24, wherein the subject has a disorder selected from a group consisting of Autism Spectrum Disorder, Schizophrenia, Rett syndrome, Down Syndrome, Tourette Syndrome, Traumatic Brain Injury, Communication, Speech, and Language Disorders, Mendelsohnn's Syndrome, Fetal Alcohol Spectrum Disorder, Fragile-X syndrome, Attention Deficit Disorder, Angelman Syndrome, Bipolar Disorder, Cerebral Palsy, Landau-Kleffner Syndrome, Phenylketonuria, Prader-Willi Syndrome, Seizure Disorder, or Williams Syndrome.

26. The method of claim 23, wherein the formulation is administered in a dose between 1 to 50 mg of ursodeoxycholic acid or analogs or conjugates or derivatives thereof per kg of bodyweight per day.
